(19) United States
(12) Patent Application Publication
(10) Pub. No.: US 2025/0013552 A1
(43) Pub. Date: Jan. 9, 2025

(54) INFORMATION PROCESSING SYSTEM FOR VISUAL FIELD EVALUATION, INFORMATION PROCESSING METHOD FOR VISUAL FIELD EVALUATION, COMPUTER PROGRAM FOR VISUAL FIELD EVALUATION, AND INFORMATION PROCESSING DEVICE

(71) Applicants: SENDAI TELEVISION INCORPORATED, Miyagi (JP); TOHOKU UNIVERSITY, Miyagi (JP)

(72) Inventors: Toru NAKAZAWA, Miyagi (JP); Takeshi YABANA, Miyagi (JP); Hiroshi KURAUCHI, Miyagi (JP); Shigeru OTA, Miyagi (JP); Shinichiro UMEMORI, Miyagi (JP); Ryoko NAKAITA, Miyagi (JP)

(73) Assignees: SENDAI TELEVISION INCORPORATED, Miyagi (JP); TOHOKU UNIVERSITY, Miyagi (JP)

(21) Appl. No.: 18/891,052

(22) Filed: Sep. 20, 2024

Related U.S. Application Data
(63) Continuation of application No. PCT/JP2023/012106, filed on Mar. 27, 2023.

(30) Foreign Application Priority Data
Apr. 1, 2022 (JP) 2022-061814

(51) Int. Cl. G06F 11/34

(52) U.S. Cl. CPC G06F 11/3466; G06F 11/3438

(57) ABSTRACT

An information processing system includes circuitry configured to control display of a visual field evaluation screen including a first, a second and a third objects, the first object being displayed in a first region set in a central portion of a display screen, the second object moving in at least a part of the first region, and the third object being displayed in at least a part of a second region for a predetermined time, perform a first determination to determine whether a positional relationship between the first and the second objects satisfies a predetermined condition in response to a first manipulation input from a user, perform a second determination to determine a second manipulation input from the user while the third object is being displayed in the second region, and generate evaluation information for a visual field of the user based on the first and the second determination.

### CROSS-REFERENCE TO RELATED APPLICATIONS

The present application is a bypass continuation of International Patent Application No. PCT/JP2023/012106, filed on Mar. 27, 2023, which claims priority to Japanese Patent Application No. 2022-061814 filed on Apr. 1, 2022, the entire disclosures of these applications are incorporated herein by reference.

### TECHNICAL FIELD

The present invention relates to an information processing system for visual field evaluation, an information processing method for visual field evaluation, an information computer program for visual field evaluation, and an information processing device.

### BACKGROUND ART

Glaucoma is a disease for which early detection is crucial. However, there is a problem that most patients with glaucoma do not experience any symptoms in the early stages, and by the time they notice abnormalities in their visual field and visit a hospital, the symptoms have often already progressed.

In recent years, a technique for creating a visual field map of a visual recognition threshold value of a subject's eye using a video game has been developed (Patent Document 1).

According to this technique, there is an advantage that a user can perform an eye examination while enjoying a game.

### CITATION LIST

### Patent Document

[Patent Document 1]
Published Japanese Translation No. 2015-502238 of the PCT International Publication

### SUMMARY OF INVENTION

### Technical Problem

However, there are still many issues with an eye examination, particularly, the evaluation of a visual field range, by a user himself or herself without the supervision of a specialist such as a doctor.

Therefore, an objective of the present invention is to provide a technique for easily obtaining evaluation information about a visual field that is easier to use and more accurate than before for contribution to the early detection of glaucoma.

### Solution to Problem

According to the present invention, there is provided an information processing system for visual field evaluation including one or more computer processors, wherein the one or more computer processors include a display processing unit configured to cause a predetermined display unit to display a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time; an input reception unit configured to receive a manipulation input from a user; a first determination unit configured to determine whether or not a positional relationship between the first object and the second object satisfies a predetermined condition when the input reception unit has received a first manipulation input from the user; a second determination unit configured to determine whether or not the input reception unit has received a second manipulation input from the user while the third object is being displayed in the second region; a generation unit configured to generate evaluation information about a visual field of the user on the basis of determination results of the first determination unit and the second determination unit; and a storage unit configured to store the evaluation information generated by the generation unit.

When the evaluation information generated by the generation unit indicates any sign of an abnormality in the visual field of the user, the display processing unit can cause the display unit to display a visual field training screen including a fourth object configured to move at least in the second region instead of the visual field evaluation screen.

The second object can allow the user to gaze at the first region, and the fourth object can allow the user to gaze at the second region.

The one or more computer processors can further include a distance information acquisition unit configured to acquire distance information about a distance between the user and the display unit; and a distance determination unit configured to determine whether or not the distance is in an appropriate range on the basis of the distance information acquired by the distance information acquisition unit. When the distance determination unit determines that the distance is not in the appropriate range, the display processing unit can display an alert screen for alerting the user to keep the distance in the appropriate range instead of the visual field evaluation screen or superimposed on the visual field evaluation screen.

The one or more computer processors can further include a size information acquisition unit configured to acquire size information about a size of the display unit, and the distance determination unit can determine whether or not the distance is in the appropriate range on the basis of the distance information acquired by the distance information acquisition unit and the size information acquired by the size information acquisition unit.

The one or more computer processors can further include an imaging unit capable of imaging a facial portion of the user, and the distance information acquisition unit can acquire the distance from an image of the facial portion of the user imaged by the imaging unit.

The one or more computer processors can further include: an imaging unit capable of imaging an eye portion of the user; and a visual field information acquisition unit configured to acquire visual line information about a visual line of the user from an image of the eye portion of the user imaged by the imaging unit.

The input reception unit can receive a touch manipulation on a first manipulation input object displayed on the visual field evaluation screen as the first manipulation input and receive a touch manipulation on a second manipulation input object displayed at a position different from that of the first manipulation input object of the visual field evaluation screen as the second manipulation input.

The display processing unit can cause the first object to be moved and displayed to follow the visual line of the user, and the input reception unit can receive a gaze operation of the user on the first object as the first manipulation input.

The one or more computer processors can further include: a sound acquisition unit capable of acquiring a sound based on an operation of the user; and a sound analysis unit configured to analyze whether the sound acquired by the sound acquisition unit corresponds to either the first manipulation input or the second manipulation input, and the input reception unit can receive the acquisition of the sound based on the operation of the user as the first manipulation input or the second manipulation input on the basis of an analysis result of the sound analysis unit.

The one or more computer processors can further include a reward giving unit configured to give a reward to the user in accordance with at least one of details of the evaluation information stored in the storage device and the number of items of the evaluation information, and the reward can be a free or discount coupon for ophthalmic evaluation by an actual doctor.

According to the present disclosure, there is provided an information processing method for visual field evaluation that causes one or more computer processors to execute: a display processing step of causing a predetermined display unit to display a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time; an input reception step of receiving a manipulation input from a user; a first determination step of determining whether or not a positional relationship between the first object and the second object satisfies a predetermined condition when a first manipulation input has been received from the user in the input reception step; a second determination step of determining whether or not a second manipulation input has been received from the user in the input reception step while the third object is being displayed in the second region; a generation step of generating evaluation information about a visual field of the user on the basis of determination results of the first determination step and the second determination step; and a storage step of storing the evaluation information generated in the generation step.

According to the present disclosure, there is provided a computer program for visual field evaluation that causes one or more computer processors to execute: a display processing function of causing a predetermined display unit to display a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time; an input reception function of receiving a manipulation input from a user; a first determination function of determining whether or not a positional relationship between the first object and the second object satisfies a predetermined condition when a first manipulation input has been received from the user in the input reception function; a second determination function of determining whether or not a second manipulation input has been received from the user in the input reception function while the third object is being displayed in the second region; a generation function of generating evaluation information about a visual field of the user on the basis of determination results of the first determination function and the second determination function; and a storage function of storing the evaluation information generated in the generation function.

According to the present disclosure, there is provided an information processing device including: a display processing unit configured to cause a predetermined display unit to display a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time; an input reception unit configured to receive a manipulation input from a user; a first determination unit configured to determine whether or not a positional relationship between the first object and the second object satisfies a predetermined condition when the input reception unit has received a first manipulation input from the user; a second determination unit configured to determine whether or not the input reception unit has received a second manipulation input from the user while the third object is being displayed in the second region; a generation unit configured to generate evaluation information about a visual field of the user on the basis of determination results of the first determination unit and the second determination unit; and a storage unit configured to store the evaluation information generated by the generation unit.

### Advantageous Effects of Invention

According to the present invention, technical improvements that solve or alleviate at least some of the problems of the conventional technique described above can be provided. Specifically, it is possible to provide an information processing system for visual field evaluation, an information processing method for visual field evaluation, an information computer program for visual field evaluation, and an information processing device that enable evaluation information about a visual field, which is easier to use and more accurate than before, to be easily obtained.

### BRIEF DESCRIPTION OF DRAWINGS

FIG. 1 is a system configuration diagram showing an example of a system configuration of an information processing system for visual field evaluation in the present disclosure.

FIG. 2 is a block diagram showing an example of a hardware configuration of an information processing device according to the present disclosure.

FIG. 3 is a functional configuration diagram showing an example of a functional configuration of an information processing system for visual field evaluation in the present disclosure.

FIG. 4 is a conceptual diagram showing an image of a screen displayed on an information processing device.

FIG. 5 is a conceptual diagram showing an image of a screen displayed on an information processing device.

FIG. 6 is a functional configuration diagram showing another example of the functional configuration of the information processing system for visual field evaluation in the present disclosure.

FIG. 7 is a conceptual diagram showing an image of a screen displayed on the information processing device.

FIG. 8 is a flowchart showing an example of a flow of an information processing method for visual field evaluation in the present disclosure.

FIG. 9 is a circuit configuration diagram showing an example of a circuit configuration for implementing a computer program for visual field evaluation in the present disclosure.

### DESCRIPTION OF EMBODIMENTS

An embodiment of an information processing system for visual field evaluation in the present disclosure will be described with reference to the drawings.

### <System Configuration>

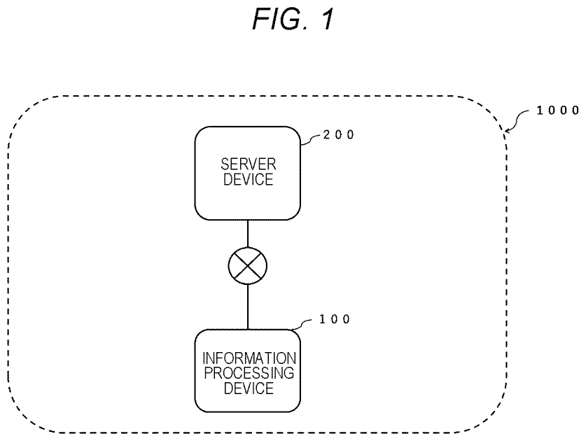

As shown in FIG. 1 as an example, an information processing system 1000 for visual field evaluation according to the present disclosure can be configured to include an information processing device 100 and a server device 200 connected to the information processing device via a network.

Alternatively, the information processing system 1000 for visual field evaluation according to the present disclosure may be configured to include only the information processing device 100.

The information processing device 100 can be a television device, a smartphone (a multifunction telephone terminal), a tablet terminal, a personal computer, a console game console, a head-mounted display (HMD), a wearable computer such as a glasses-type wearable terminal (AR glasses or the like), and an information processing device capable of reproducing moving images other than these devices. Moreover, these terminals may be stand-alone devices that operate alone or may include a plurality of devices connected so that various types of data can be transmitted and received to and from each other.

### <Hardware Configuration>

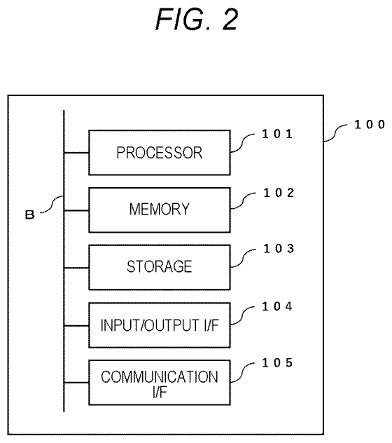

Here, a hardware configuration of the information processing device 100 will be described with reference to FIG. 2. The information processing device 100 can include a processor 101, a memory 102, a storage 103, an input/output interface (input/output I/F) 104, and a communication interface (communication I/F) 105. The constituent elements are interconnected via a bus B.

The information processing device 100 can implement functions and methods to be described in the present embodiment by cooperating with the processor 101, the memory 102, the storage 103, the input/output I/F 104, and the communication I/F 105.

The processor 101 executes functions and/or methods implemented by codes or instructions included in programs stored in the storage 103. The processor 101 may include, for example, a central processing unit (CPU), a micro processing unit (MPU), a graphics processing unit (GPU), a microprocessor, a processor core, a multiprocessor, an application-specific integrated circuit (ASIC), a field programmable gate array (FPGA), and the like and logic circuits (hardware) and dedicated circuits formed in integrated circuits (an integrated circuit (IC) chip and a large-scale integration (LSI) circuit) and the like may implement processes disclosed in the embodiments. Also, these circuits may be implemented by one or more integrated circuits and a plurality of processes shown in the embodiments may be implemented by one integrated circuit. Also, the LSI circuit may be referred to as a very large-scale integration (VLSI) circuit, a super-LSI circuit, an ultra-LSI circuit, or the like according to an integration degree difference.

The memory 102 temporarily stores programs loaded from the storage 103 and provides a work area for the processor 101. The memory 102 also temporarily stores various types of data generated while the processor 101 is executing the program. The memory 102 includes, for example, a random-access memory (RAM), a read-only memory (ROM), and the like.

The storage 103 stores programs. The storage 103 includes, for example, a hard disk drive (HDD), a solid-state drive (SSD), a flash memory, and the like.

The communication I/F 105 is implemented as hardware such as a network adapter, communication software, or a combination thereof and transmits and receives various types of data via a network. The communication may be performed by wire or wirelessly and any communication protocol may be used as long as mutual communication can be executed. The communication I/F 105 communicates with other information processing devices via the network. The communication I/F 105 transmits various types of data to other information processing devices in accordance with an instruction from the processor 101. Also, the communication I/F 105 also receives various types of data transmitted from other information processing devices and transmits the data to the processor 101.

The input/output I/F 104 includes an input device for inputting various types of manipulations to the information processing device 100 and an output device for outputting a processing result after a process performed by the information processing device 100. The input/output I/F 104 may be integrated with the input device and the output device or may be separated into the input device and the output device.

The input device is implemented by any one or a combination of all types of devices that can receive an input from the user and transmit information related to the input to the processor 101. The input device includes, for example, hardware keys of a touch panel, a touch display, and a keyboard, a pointing device such as a mouse, a camera (a manipulation input via an image), and a microphone (a manipulation input by sound).

Moreover, the server device 200 in the present disclosure can also include a hardware configuration as in FIG. 2, unless otherwise specified.

### Embodiment

Next, various types of functions capable of being executed by one or more computer processors provided in the information processing system 1000 for visual field evaluation according to the embodiment of the present disclosure will be described with reference to the drawings.

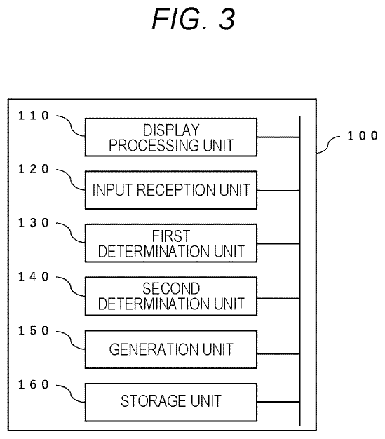

As shown in FIG. 3, one or more computer processors provided in the information processing system 1000 for visual field evaluation in the present disclosure include a display processing unit 110, an input reception unit 120, a first determination unit 130, a second determination unit 140, a generation unit 150, and a storage unit 160.

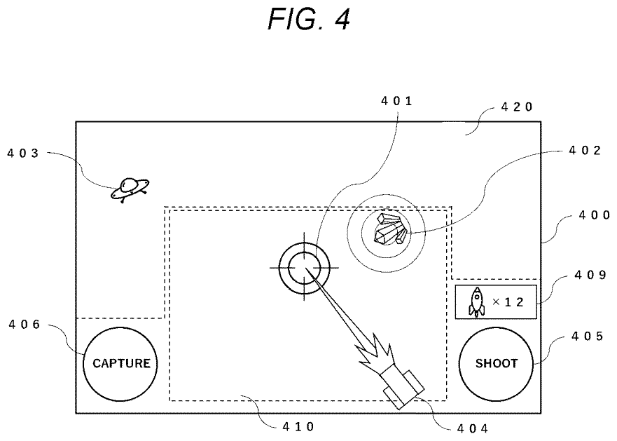

FIG. 4 shows an image of a visual field evaluation screen 400 displayed in a display unit that is a display provided in the information processing device 100 or connected to the information processing device 100 by wire or wirelessly.

As shown as an example in FIG. 4, the display processing unit 110 causes a predetermined display unit to display the visual field evaluation screen 400 including a first object 401, a second object 402, and a third object 403.

The first object 401 is displayed in a first region 410 set in a central portion of the displayed screen 400.

As an example, the first object 401 can indicate an aiming object when an object such as a bullet is fired from an object 404 such as a missile. In addition, it is assumed that the first object 401 is fixed and displayed in the central portion of the displayed screen 400.

This first object 401 is displayed to fix a visual line of a user. Therefore, the first object 401 is displayed to be fixed to the center of the displayed screen 400.

The second object 402 is different from the first object 401 and moves at least in the first region 410.

As an example, the second object 402 can be an object such as a meteorite. Also, it is only necessary for the second object 402 to move at least in the first region 410 and the second object 402 may move from outside the first region 410 to inside the first region 410. There is no particular restriction on a route of movement.

The first region 410 is set in the central portion of the displayed screen 400, and its shape is not particularly limited. As an example, in FIG. 4, a rectangular region is set as the first region 410.

The third object 403 is different from the first object 401 and the second object 402 and is displayed at least in a second region 420 outside the first region 410 for a predetermined time.

As an example, the third object 403 can be an object of a capture target. Also, the third object 403 is displayed in the second region 420 for a predetermined time.

The second region 420 is set in an outer circumferential portion of the displayed screen 400 and its shape is not particularly limited. As an example, in FIG. 4, a rectangular region surrounding the first region 410 is set as the second region 420.

In addition, in the first region 410 and the second region 420 described above, these boundaries may be visible or invisible.

The time during which the third object 403 is displayed is not particularly limited, and can be several seconds as an example.

Moreover, the third object 403 may be displayed anywhere in the second region 420, but it is assumed that the third object 403 is displayed to be fixed without moving after the display.

The input reception unit 120 receives a manipulation input from the user.

When the information processing device 100 is a terminal having a touch panel such as a smartphone or a tablet terminal and is used at a predetermined distance from the user, a manipulation input from the user can be a touch manipulation on the touch panel.

Moreover, regardless of whether or not there is a touch panel of the information processing device 100, the manipulation input from the user can be a button pressing manipulation for a controller, a remote control, or the like connected to the information processing device 100 by wire or wirelessly.

Moreover, regardless of whether or not there is a touch panel of the information processing device 100, the manipulation input from the user may be based on others such as a sound and a visual line. Details will be described below.

Moreover, even if the information processing device 100 is a terminal having a touch panel, when the information processing device 100 is not used at a predetermined distance from the user (when a smartphone is used as an HMD display as an example), a manipulation input based on a manipulation for pressing the button or the like can be applied.

In addition, a manipulation input from the user will be described as a touch manipulation on the touch panel in the following example.

The first determination unit 130 determines whether or not a positional relationship between the first object 401 and the second object 402 satisfies a predetermined condition when the input reception unit 120 has received the first manipulation input from the user.

As an example, the input reception unit 120 receives a touch manipulation on a first manipulation input object 405 displayed on the visual field evaluation screen 400 as the first manipulation input.

In FIG. 4, an object having the text “SHOOT” is shown as the first manipulation input object 405.

As an example, a predetermined condition can be that a distance between coordinates of a central position of the first object 401 and coordinates of a central position of the second object 402 is less than or equal to a predetermined value. Moreover, coordinates of an outer edge position of the object may be used for the predetermined condition.

In other words, the first determination unit 130 determines whether or not a bullet has hit the meteorite.

The second determination unit 140 determines whether or not the input reception unit 120 has received a second manipulation input from the user while the third object 403 is being displayed in the second region 420.

As an example, the input reception unit 120 receives a touch manipulation on a second manipulation input object 406 displayed at a position different from that of the first manipulation input object 405 of the visual field evaluation screen 400 as the second manipulation input.

In FIG. 4, an object having the text “CAPTURE” is shown as the second manipulation input object 406.

Specifically, the second determination unit 140 determines whether or not the capture target has been able to be captured.

The generation unit 150 generates evaluation information about a visual field of the user on the basis of determination results of the first determination unit 130 and the second determination unit 140.

In a game for displaying the visual field evaluation screen in the present disclosure, the user is allowed to concentrate on a task of shooting down a meteorite in the first region 410 set in the central portion of the display screen. This is to fix the center of the user's visual field and a result of the first determination unit 130 can be used to determine whether the user has appropriately fixed the center of the visual field.

Also, in the game for displaying the visual field evaluation screen in the present disclosure, the user is required to perform a task of capturing a capture object appearing at an outer circumferential portion of the display screen, i.e., at an edge of the visual field. This is to confirm a range of the user's visual field and the success of the capture indicates that the capture target has been located and recognized in the user's visual field.

However, if the capture is successful but the meteorite is not successfully shot down, there is a possibility that the center of the user's visual field may not be fixed to the central portion of the display screen.

Therefore, evaluation information about the user's visual field is generated on the basis of the determination results of both the first determination unit 130 and the second determination unit 140.

Specifically, in the beginning, a first success rate is calculated on the basis of the determination result of the first determination unit 130. As an example, in the first success rate, the number of times a positional relationship between the first object 401 and the second object 402 satisfies a predetermined condition can be designated as a denominator and the number of times the first manipulation input has been received in a state in which the positional relationship between the first object 401 and the second object 402 satisfies the predetermined condition can be designated as a numerator.

In addition, the first value is not particularly limited and can be set to 80% as an example.

Also, when the first success rate is greater than or equal to the first value, a second success rate is calculated on the basis of the determination result of the second determination unit 140. As an example, in the second success rate, the number of times the third object 403 has been displayed in the second region 420 can be designated as a denominator and the number of times the second manipulation input has been received in a state in which the third object 403 is displayed in the second region 420 can be designated as a numerator.

On the other hand, when the first success rate is less than the first value, a screen for prompting the user to play the game again can be displayed.

Also, as an example, the evaluation information about the user's visual field can be a ratio of the second success rate to the first success rate. When this ratio is low, there is a high possibility that the user will not be able to look at an end of the visual field and it can be evaluated that there is any sign of an abnormality in the user's visual field.

On the other hand, when the above ratio is high, there is a high possibility that the user will be able to look at an end of the visual field and it can be evaluated that there is no sign of an abnormality in the user's visual field.

In addition, the evaluation information about the user's visual field may include not only the above ratio but also the second success rate itself when the first success rate is greater than or equal to the first value. Moreover, a failure rate may be calculated and used instead of the success rate.

The storage unit 160 stores evaluation information generated by the generation unit 150.

In addition, the evaluation information can be stored in association with user information about the user. The user information can include information such as the user's name, date of birth, age, gender, lifestyle habits, and visual acuity.

Although one or more computer processors provided in the information processing device 100 include the display processing unit 110, the input reception unit 120, the first determination unit 130, and the second determination unit 140 and one or more computer processors provided in the server device 200 include the generation unit 150 and the storage unit 160 in the present embodiment, the present disclosure is not limited thereto. All computer processors may be provided in the information processing device 100. In this case, the information processing system does not need to include the server device 200.

According to the above configuration, it is possible to obtain evaluation information about a visual field that is easier to use and more accurate than before.

When the evaluation information generated by the generation unit 150 indicates any sign of an abnormality in the user's visual field, the display processing unit 110 can cause the display unit to display a visual field training screen 500 instead of the visual field evaluation screen 400.

As described above, a case where the evaluation information indicates any sign of an abnormality in the user's visual field can be a case where the second success rate is less than a second value, a case where the above ratio is less than a third value, or the like. These second and third values can be appropriately determined by a specialist such as a doctor.

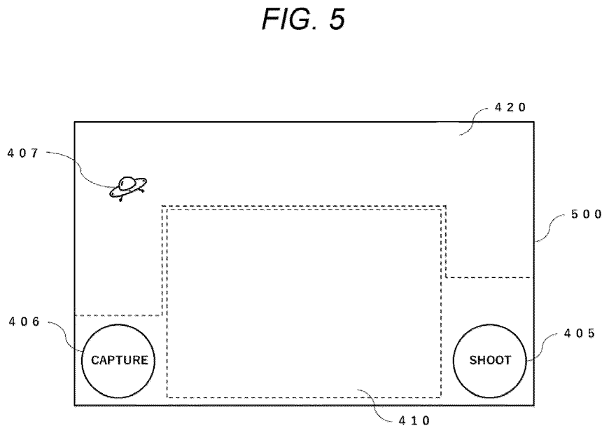

FIG. 5 shows an image of the visual field training screen 500 displayed on the display unit.

As shown as an example in FIG. 5, the display processing unit 110 causes the display unit to display the visual field training screen 500 including a fourth object 407.

The fourth object 407 is displayed at least in the second region 420.

In addition, the fourth object 407 may be the above-described third object 403. The fourth object 407 may be displayed in the second region 420 for a predetermined time and displayed to be fixed without moving after the display or may move during the display.

In this case, the determination process of the first determination unit 130 is not performed, but the determination process of the second determination unit 140 is performed.

A difference between the visual field evaluation screen 400 and the visual field training screen 500 described above can be at least the presence or absence of the display of the first object 401 and the second object 402 in the first region 410.

That is, the first object 401 and the second object 402 are used to allow the user to gaze at the first region 410 and the fourth object 407 is used to allow the user to gaze at the second region 420.

According to the above configuration, when it is evaluated that there is any sign of an abnormality in the user's visual field, a training process can be performed to improve the abnormality in the visual field or prevent its worsening.

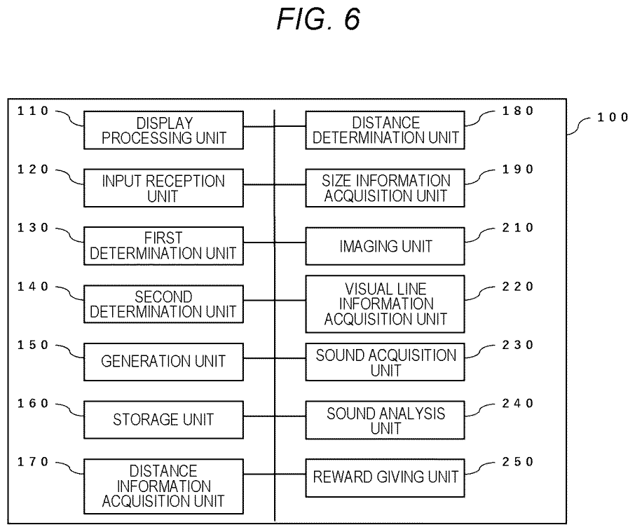

Moreover, as shown in FIG. 6 as an example, the one or more computer processors can further include a distance information acquisition unit 170 and a distance determination unit 180.

The distance information acquisition unit 170 acquires distance information about a distance between the user and the display unit.

Although the distance information may be obtained by estimating a distance between the user and the display unit from a size of the user's face or eyes captured using an imaging function provided in the information processing device 100 as an example, the present disclosure is not limited thereto. Various well-known distance estimation techniques can be applied.

The distance determination unit 180 determines whether or not the distance is in an appropriate range on the basis of the distance information acquired by the distance information acquisition unit 170.

Also, when the distance determination unit 180 determines that the distance is not in the appropriate range, the display processing unit 110 displays an alert screen.

The alert screen for alerting the user to keep the distance in the appropriate range instead of the visual field evaluation screen 400 or superimposed on the visual field evaluation screen 400 is used.

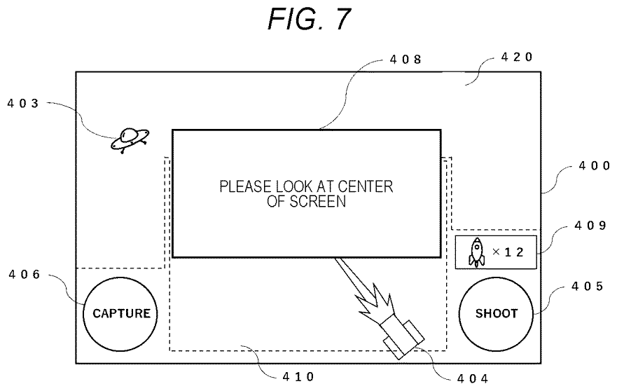

FIG. 7 shows an example of an alert screen 408 displayed to be superimposed on the visual field evaluation screen 400.

Moreover, as shown in FIG. 6 as an example, the one or more computer processors can further include a size information acquisition unit 190.

The size information acquisition unit 190 acquires size information about a size of the display unit.

At this time, the distance determination unit 180 determines whether or not the distance is in an appropriate range on the basis of distance information acquired by the distance information acquisition unit 170 and size information acquired by the size information acquisition unit 190.

Specifically, the user needs to be closer to the display unit when the size of the display unit is smaller and the user needs to be farther from the display unit when the size of the display unit is larger.

Moreover, as shown in FIG. 6 as an example, the one or more computer processors can further include an imaging unit 210.

The imaging unit 210 is capable of imaging the user's face.

Also, as described above, the distance information acquisition unit 170 acquires a distance from the image of the user's face captured by the imaging unit 210.

Moreover, the imaging unit 210 may capture an image of the user's eye.

At this time, as shown in FIG. 6 as an example, the one or more computer processors can further include a visual line information acquisition unit 220.

The visual line information acquisition unit 220 acquires visual line information about the user's visual line from an image of the user's eye portion captured by the imaging unit 210.

The visual line information can be used for a manipulation input of the user as described below.

As described above, the above-described input reception unit 120 can receive a touch manipulation on the first manipulation input object 405 displayed on the visual field evaluation screen 400 as a first manipulation input and can receive a touch manipulation on the second manipulation input object 406 displayed at a position different from that of the first manipulation input object of the visual field evaluation screen 400 as a second manipulation input.

Moreover, the display processing unit 110 may cause the first object 401 to be moved and displayed to follow the user's visual line and the input reception unit 120 may receive the user's gaze operation on the first object 401 as the first manipulation input.

However, it is assumed that a range in which the first object 401 moves while following the user's visual line is limited to the central portion of the screen 400.

Moreover, as shown in FIG. 6 as an example, the one or more computer processors can further include a sound acquisition unit 230 and a sound analysis unit 240.

The sound acquisition unit 230 is capable of acquiring a sound based on the user's operation.

The sound analysis unit 240 analyzes whether the sound acquired by the sound acquisition unit 230 corresponds to the first manipulation input or the second manipulation input.

In addition, a sound acquisition technique and a sound analysis technique are not particularly limited and various well-known techniques can be applied.

Also, it is assumed that the input reception unit 120 receives the acquisition of a sound based on the user's operation as the first manipulation input or the second manipulation input on the basis of an analysis result of the sound analysis unit 240.

As an example, it can be assumed that the sound analysis unit 240 receives the utterance “SHOOT” from the user as the first manipulation input and receives the utterance “CAPTURE” from the user as the first manipulation input.

As another example, it can be assumed that the sound analysis unit 240 receives a sound of the user clapping his or her hand once as the first manipulation input and receives a sound of the user clapping his or her hand twice as the first manipulation input.

In addition, the sound corresponding to the manipulation input is not limited to the above example, may be another sound, or may be a sound capable of being set by the user himself or herself.

Moreover, as shown in FIG. 6 as an example, the one or more computer processors may further include a reward giving unit 250.

The reward giving unit 250 gives a reward to the user in accordance with details of the evaluation information and/or the number of evaluation information items stored in the storage unit 160.

The reward may be free or discount coupons for examinations by actual doctors and other specialists. Such a reward can be given electronically as electronic data.

According to the above configuration, even if the user is unable to perform a manipulation using his or her hands, it is possible to perform a manipulation according to a sound input.

### Other Embodiments

As shown in FIG. 4 and the like, the visual field evaluation screen 400 may include a display item 409 indicating the remaining number of bullets. It is assumed that the game will end and the above-described success rate and other factors are tabulated if the remaining number of bullets reaches zero.

Moreover, the visual field evaluation game implemented by the invention of the present disclosure is a game in which a meteorite appearing irregularly in the center is destroyed with a SHOOT button and ores scattered in the vicinity and the like are collected as an example. The user can play until the time limit expires or the specified number of errors is reached (the remaining bullets run out). The meteorite in the center is used to maintain a viewpoint in the center and a task of collecting ores in the vicinity is performed to determine an angle of a visual field. It is also possible to accumulate play logs of the user, perform data analysis, calculate the characteristics and risks of each user, and automatically adjust (optimize) a difficulty level and give advice. In order to increase the user's motivation to play regularly, it is also possible to add an external linkage service such as issuing coupons according to the score.

If the user successfully taps the SHOOT button when there are a cursor (the first object) in the center and a meteorite (the second object), it is possible to obtain points. On the other hand, if the user fails to tap, the number of bullets will decrease.

Moreover, a capture icon (the third object) appears for a certain time and a score is increased if the user can perform a capture process. Even if there is a failure, the number of points is not reduced, and it is assumed that the failure is recorded as a manipulation log.

Subsequently, an information processing method for visual field evaluation in the embodiment of the present disclosure will be described. The information processing method for visual field evaluation is an information processing method for visual field evaluation that is executed by the above-described information processing system 1000 for visual field evaluation.

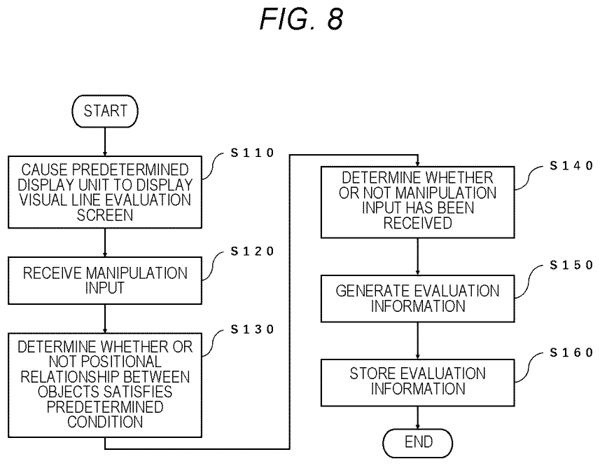

As shown in FIG. 8 as an example, the information processing method for visual field evaluation in the present disclosure is characterized in that a display processing step S110, an input reception step S120, a first determination step S130, a second determination step S140, a generation step S150, and a storage step S160 are executed by the one or more computer processors.

In the display processing step S110, a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time is displayed on a predetermined display unit. This display processing step S110 can be executed by the display processing unit 110 described above.

In the input reception step S120, a manipulation input from the user is received. The input reception step S120 can be executed by the input reception unit 120 described above.

In the first determination step S130, it is determined whether or not a positional relationship between the first object and the second object satisfies a predetermined condition when the first manipulation input from the user has been received in the input reception step S120. This first determination step S130 can be executed by the above-described first determination unit 130.

In the second determination step S140, it is determined whether or not the second manipulation input from the user has been received in the input reception step while the third object is being displayed in the second region. This second determination step S140 can be executed by the above-described second determination unit 140.

In the generation step S150, evaluation information about the user's visual field is generated on the basis of determination results of the first determination step S130 and the second determination step S140. This generation step S150 can be executed by the above-described generation unit 150.

In the storage step S160, the evaluation information generated in the generation step S150 is stored. This storage step S160 can be executed by the above-described storage unit 160.

According to the above configuration, evaluation information about the visual field that is easier to use and more accurate than before can be obtained.

Subsequently, a computer program in an embodiment of the present disclosure will be described. This computer program is a computer program that is executed in the information processing system 1000 for visual field evaluation described above.

The computer program in the present disclosure is characterized in that one or more computer processors implement a display processing function, an input reception function, a first determination function, a second determination function, a generation function, and a storage function.

In the display processing function, a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time is displayed on a predetermined display unit.

In the input reception function, a manipulation input from the user is received.

In the first determination function, it is determined whether or not a positional relationship between the first object and the second object satisfies a predetermined condition when a first manipulation input from the user has been received in the input reception function.

In the second determination function, it is determined whether or not a second manipulation input from the user has been received in the input reception function while the third object is being displayed in the second region.

In the generation function, evaluation information about the user's visual field is generated on the basis of determination results of the first determination function and the second determination function.

In the storage function, the evaluation information generated in the generation function is stored.

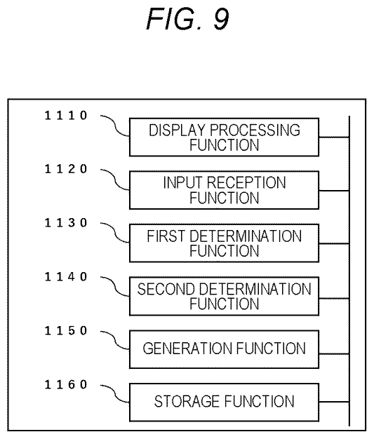

The above-described functions can be implemented by a display processing circuit 1110, an input reception circuit 1120, a first determination circuit 1130, a second determination circuit 1140, a generation circuit 1150, and a storage circuit 1160 shown in FIG. 9. It is assumed that the input reception circuit 1120, the first determination circuit 1130, the second determination circuit 1140, the generation circuit 1150, and the storage circuit 1160 are implemented by the display processing unit 110, the input reception unit 120, the first determination unit 130, the second determination unit 140, the generation unit 150, and the storage unit 160 described above. Details of each part have been described above.

According to the above configuration, evaluation information about a visual field that is easier to use and more accurate than before can be obtained.

Finally, the information processing device in the embodiment of the present disclosure will be described. This information processing device is the information processing device 100 in the above-described information processing system 1000 for visual field evaluation.

As shown as an example in FIG. 3, the information processing device in the present disclosure is characterized in that the display processing unit 110, the input reception unit 120, the first determination unit 130, the second determination unit 140, the generation unit 150, and the storage unit 160 are provided therein.

The display processing unit 110 causes a predetermined display unit to display a visual field evaluation screen including a first object displayed in a first region set in a central portion of the displayed screen, a second object different from the first object and configured to move at least in the first region, and a third object different from the first object and the second object and configured to be displayed at least in a second region outside the first region for a predetermined time. Details of this display processing unit 110 are as described above.

The input reception unit 120 receives a manipulation input from the user. Details of this input reception unit 120 are as described above.

The first determination unit 130 determines whether or not a positional relationship between the first object and the second object satisfies a predetermined condition when the input reception unit 120 has received a first manipulation input from the user. Details of this first determination unit 130 are as described above.

The second determination unit 140 determines whether or not the input reception unit 120 has received a second manipulation input from the user while the third object is being displayed in the second region. Details of this second determination unit 140 are as described above.

The generation unit 150 generates evaluation information about the user's visual field on the basis of determination results of the first determination unit 130 and the second determination unit 140. Details of this generation unit 150 are as described above.

The storage unit 160 stores evaluation information generated by the generation unit 150. Details of this storage unit 160 are as described above.

According to the above configuration, evaluation information about a visual field that is easier to use and more accurate than before can be obtained.

Moreover, an information processing device such as a computer or a mobile phone can be suitably used to function as a server device or a terminal device according to the above-described embodiment. This information processing device can be implemented by storing a program describing processing content for implementing each function of the server device or the terminal device according to the embodiment in the storage unit of the information processing device and reading and executing the program with a CPU of the information processing device.

While several embodiments of the present invention have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. These embodiments and their modifications are included in the scope and spirit of the inventions and included in the scope of the inventions described in the accompanying claims and its equivalents.

Moreover, the method described in the embodiment can be stored as a program capable of being executed by a computer in a recording medium, for example, such as a magnetic disk (a floppy (registered trademark) disk, a hard disk, or the like), an optical disc (a compact disc (CD)-ROM, a digital versatile disc (DVD), a magneto-optical disc (MO), or the like), or a semiconductor memory (a ROM, a RAM, a flash memory, or the like), or can be transmitted and distributed via a communication medium. The program stored in the medium also includes a setup program for configuring software means (including not only execution programs but also tables and data structures) to be executed by the computer in the computer. A computer that implements this device reads a program recorded on a recording medium, and constructs software means by a setup program in some cases, and executes the above-described process by controlling the operation using the software means. The term “recording medium” described here is not limited to those for distribution, and includes storage media such as magnetic disks and semiconductor memories provided in computers or devices connected via a network. The storage unit may function, for example, as a main storage device, an auxiliary storage device, or a cache memory.

### REFERENCE SIGNS LIST

100 Information processing device
110 Display processing unit
120 Input reception unit
130 First determination unit
140 Second determination unit
150 Generation unit
160 Storage unit
200 Server device
1000 Information processing system for visual field evaluation

1. An information processing system for visual field evaluation comprising:
circuitry configured to
control a display to display a visual field evaluation screen including a first object, a second object and a third object, the first object being displayed in a first region set in a central portion of a display screen, the second object moving in at least a part of the first region and being different from the first object, and the third object being displayed in at least a part of a second region outside the first region in the display screen for a predetermined time, and being different from the first object and the second object,
perform a first determination to determine whether or not a positional relationship between the first object and the second object satisfies a predetermined condition, in response to a first manipulation input from a user,
perform a second determination to determine a second manipulation input from the user while the third object is being displayed in the second region, and
generate evaluation information for a visual field of the user on the basis of determination results of the first determination and the second determination unit.

2. The information processing system for visual field evaluation according to claim 1, wherein, under a condition that the evaluation information indicates a sign of an abnormality in the visual field of the user,
the circuitry is configured to control the display to display a visual field training screen including a fourth object that moves in at least a part of the second region instead of the visual field evaluation screen.

3. The information processing system for visual field evaluation according to claim 2,
wherein the second object is arranged to gaze at the first region, and
wherein the fourth object is arranged the user to gaze at the second region.

4. The information processing system for visual field evaluation according to claim 1,
wherein the circuitry is further
configured to acquire distance information for a distance between the user and the display,
wherein the circuitry is further configured to determine whether or not the distance is in an appropriate range on the basis of the distance information, and
wherein, under a condition that the distance is not in the appropriate range,
the circuitry is configured to control the display to display an alert screen for alerting the user to keep the distance in the appropriate range, by replacing the visual field evaluation screen with the alert screen, or by superimposing the alert screen on the visual field evaluation screen.

5. The information processing system for visual field evaluation according to claim 4,
wherein the circuitry is further configured to acquire size information for a size of the display, and
wherein the circuitry is configured to determine whether or not the distance is in the appropriate range on the basis of the distance information and the size information.

6. The information processing system for visual field evaluation according to claim 4, further comprising:
a camera to capture an image of a facial portion of the user, and
wherein the circuitry is configured to acquire the distance from the image of the facial portion of the user imaged by the camera.

7. The information processing system for visual field evaluation according to claim 1, further comprising:
a camera to capture an image of an eye portion of the user; and
wherein the circuitry is configured to acquire visual line information of a visual line of the user from the image of the eye portion of the user imaged by the camera.

8. The information processing system for visual field evaluation according to claim 1, wherein the circuitry is configured to detect a touch manipulation on a first manipulation input object displayed on the visual field evaluation screen as the first manipulation input and detect a touch manipulation on a second manipulation input object displayed at a position different from that of the first manipulation input object of the visual field evaluation screen as the second manipulation input.

9. The information processing system for visual field evaluation according to claim 7,
wherein the circuitry is configured to control the display to display the first object to be moved and displayed to follow the visual line of the user, and
wherein the circuitry is configured to control reception of a gaze operation of the user on the first object as the first manipulation input.

10. The information processing system for visual field evaluation according to claim 1,
wherein the circuitry is further configured to acquire
a sound based on an operation of the user; and
wherein the circuitry is further configured to analyze whether the acquired sound corresponds to either the first manipulation input or the second manipulation input, and
wherein the circuitry is configured to control reception of the acquisition of the sound based on the operation of the user as the first manipulation input or the second manipulation input on the basis of the analysis result whether the acquired sound corresponds to either the first manipulation input or the second manipulation input.

11. The information processing system for visual field evaluation according to claim 1,
wherein the circuitry is further configured to give a reward to the user in accordance with at least one of content of the evaluation information and a number of items of the evaluation information, and
wherein the reward is a free or discount coupon for ophthalmic evaluation by an actual doctor.

12. An information processing method for visual field evaluation comprising:
controlling a display to display a visual field evaluation screen including a first object, a second object and a third object, the first object being displayed in a first region set in a central portion of a display screen, the second object moving in at least a part of the first region and being different from the first object, and the third object being displayed in at least a part of a second region outside the first region in the display screen for a predetermined time, and being different from the first object and the second object;
performing a first determination to determine whether or not a positional relationship between the first object and the second object satisfies a predetermined condition, in response to a first manipulation input from a user;
performing a second determination to determine a second manipulation input from the user while the third object is being displayed in the second region; and
generating evaluation information for a visual field of the user on the basis of determination results of the first determination and the second determination.

13. A non-transitory computer-readable recording medium storing a computer executable program which, when executed by a computer, cause the computer to:
controlling a display to display a visual field evaluation screen including a first object, a second object and a third object, the first object being displayed in a first region set in a central portion of a display screen, the second object moving in at least a part of the first region and being different from the first object, and the third object being displayed in at least a part of a second region outside the first region in the display screen for a predetermined time, and being different from the first object and the second object;
performing a first determination to determine whether or not a positional relationship between the first object and the second object satisfies a predetermined condition, in response to a first manipulation input from a user;
performing a second determination to determine a second manipulation input from the user while the third object is being displayed in the second region; and
generating evaluation information for a visual field of the user on the basis of determination results of the first determination and the second determination.

14. An information processing device comprising:
a display to display a visual field evaluation screen;
a memory to store computer executable program and information; and
circuitry configured to
control the display to display the visual field evaluation screen including a first object, a second object and a third object, the first object being displayed in a first region set in a central portion of a display screen, the second object moving in at least a part of the first region and being different from the first object, and the third object being displayed in at least a part of a second region outside the first region in the display screen for a predetermined time, and being different from the first object and the second object,
perform a first determination to determine whether or not a positional relationship between the first object and the second object satisfies a predetermined condition, in response to a first manipulation input from a user,
perform a second determination to determine a second manipulation input from the user while the third object is being displayed in the second region, and
generate evaluation information for a visual field of the user on the basis of determination results of the first determination and the second determination.
